(19) United States
(12) Patent Application Publication
(10) Pub. No.: US 2013/0281924 A1
(43) Pub. Date: Oct. 24, 2013

(54) Segmented instrument shaft with antirotation features

(71) Applicant: TransEnterix, Inc., Larkspur, CA (US)

(72) Inventor: Carson Shellenberger, Raleigh, NC (US)

(21) Appl. No.: 13/651,278

(22) Filed: Oct. 12, 2012

Related U.S. Application Data
(63) Continuation-in-part of application No. 12/846,804, filed on Jul. 29, 2010.
Application No. 12/846,804 is a continuation of application No. PCT/US2011/130457, filed on Apr. 13, 2011.
(60) Provisional application No. 61/323,863, filed on Apr. 13, 2010.

(51) Int. Cl. A61M 25/01

(52) U.S. Cl. CPC A61M 25/0147
USPC 604/95.01

(57) ABSTRACT

Deflectable instrument shafts are formed of a plurality of vertebral segments. One embodiment utilizes alternating segments, each of which has a first end or face contacting an adjacent segment along a first plane, and a second end or face contacting an adjacent segment along a second plane that is transverse to the first plane. In some embodiments, the alternating segments are first and second segments having differently shaped contacting ends/faces. In other embodiment the alternating segments are identical to one another but are positioned such that segments having their first contacting end/face facing distally are alternated with segments having their second contacting ends/faces facing distally. In other embodiments, embodiments having semi-spherical articulating surfaces are used.

This application is a continuation of PCT/US2011/130457, filed Apr. 13, 2011, which claims the benefit of U.S. Provisional Application No. 61/323,863, filed Apr. 13, 2010, which is incorporated herein by reference. This application is also a continuation-in-part of U.S. application Ser. No. 12/846,804, filed Jul. 29, 2010. Each of the foregoing patent applications is incorporated herein by reference.

### TECHNICAL FIELD OF THE INVENTION

The present invention relates generally to the field of actively deflectable shafts for medical devices such as instruments or instrument access devices.

### BACKGROUND

Surgery in the abdominal cavity is frequently performed using open laparoscopic procedures, in which multiple small incisions or ports are formed through the skin and underlying muscle and peritoneal tissue to gain access to the peritoneal site using the various instruments and scopes needed to complete the procedure. The peritoneal cavity is typically inflated using insufflation gas to expand the cavity, thus improving visualization and working space. Further developments have lead to systems allowing such procedures to be performed using only a single port.

In single port surgery (“SPS”) procedures, it is useful to position an access device within the incision to give access to the operative space without loss of insufflation pressure. Ideally, such a device provides sealed access for multiple instruments while avoiding conflict between instruments during their simultaneous use. Some multi-instrument access devices or ports suitable for use in SPS procedures and other laparoscopic procedures are described in co-pending U.S. application Ser. No. 11/804,063 (063 application) filed May 17, 2007 and entitled SYSTEM AND METHOD FOR MULTI-INSTRUMENT SURGICAL ACCESS USING A SINGLE ACCESS PORT, U.S. application Ser. No. 12/209,408 filed Sep. 12, 2008 and entitled MULTI-INSTRUMENT ACCESS DEVICES AND SYSTEMS, and U.S. application Ser. No. 12/511,043 (Attorney Docket No. TRX-2220), filed Jul. 28, 2009, entitled MULTI-INSTRUMENT ACCESS DEVICES AND SYSTEMS, and U.S. application Ser. No. 12/846,788 (Attorney Docket No. TRX-2520, entitled DEFLECTABLE INSTRUMENT PORTS, filed Jul. 29, 2010, each of which is incorporated herein by reference. The aforementioned patent applications describe access systems incorporating at least one and preferably multiple instrument delivery tubes having deflectable distal ends. Deflection or steering of flexible instruments passed through the instrument delivery tubes is carried out using the deflectable instrument delivery tubes. The present application describes embodiments of instrument delivery tube shafts that may be used for this purpose, or that may be used with other single- or multi-instrument trocars, access ports, or intravascular access systems including those known to those skilled in the art.

### BRIEF DESCRIPTION OF THE DRAWINGS

FIG. 1 is a perspective view showing the distal end portion of a first embodiment of a deflectable shaft;

FIG. 2A is a side elevation view of two segments of the embodiment of FIG. 1;

FIG. 2B is similar to FIG. 2A but shows the assembly axially rotated by forty-five degrees;

FIG. 3A is a plan view of the distal end of the first segment of FIG. 2A;

FIG. 3B is a plan view of the proximal end of the first segment of FIG. 2A;

FIG. 4A is a plan view of the distal end of the second segment of FIG. 2A;

FIG. 4B is a plan view of the proximal end of the second segment of FIG. 2A;

FIG. 5A shows the distal end portion of FIG. 1 in a curved position;

FIG. 5B shows two of the segments of FIG. 5A;

FIGS. 6A and 6B are perspective views of one type of surgical access system employing instrument delivery tubes with shafts of the type shown in FIG. 1. FIG. 6A shows the instrument delivery tubes in a straight and side-by-side arrangement for deployment. FIG. 6B shows the instrument delivery tubes laterally separated for use and deflected into a curve.

FIG. 7 is a perspective view showing a distal end section of a second embodiment of an instrument delivery tube. In this figure the instrument delivery tube is shown deflected into a curve.

FIGS. 8A, 8B and 8C are a proximal plan view, a side elevation view, and proximal a perspective view, respectively, of a first segment of the embodiment of FIG. 7.

FIGS. 9A, 9B and 9C are a distal plan view, a side elevation view, and a distal perspective view, respectively, of a second segment of the embodiment of FIG. 7.

FIG. 10 is a perspective view showing, in a deflected position, the distal end portion of a third embodiment of a deflectable shaft.

FIGS. 11A-11E are a collection of views of one of the segments of the embodiment of FIG. 10, in which FIG. 11A is a side elevation view, FIG. 11B is a plan view, FIG. 11C is a side elevation view, FIG. 11D is a cross-section view taken along plane A-A of FIG. 11C, and FIG. 11E is a perspective view.

FIG. 12a is a perspective view of a fourth embodiment of a deflectable shaft.

FIG. 12b is an enlarged view of the distal section and intermediate member of the fourth embodiment.

FIG. 12c is an enlarged view of the proximal section and intermediate member of the fourth embodiment.

FIG. 12d is a perspective view showing the fourth embodiment in a deflected position.

FIG. 13 is a perspective view of a fifth embodiment of a deflectable shaft shown on an instrument delivery tube.

FIG. 14 is a partially exploded view of the distal end portion of the shaft FIG. 13.

FIG. 15A is a partially exploded perspective view of three of the segments of FIG. 14, in which two segments are assembled and a third segment is positioned for assembly.

FIG. 15B is a perspective view of a rigid segment of the shaft FIG. 14.

FIG. 16 is a plan view of alternative segments that may be used to form a shaft, and further illustrates positioning of the pull elements.

FIG. 17A is a side elevation view of an alternative to the fifth embodiment.

FIG. 17B is a plan view of a wave spring of the embodiment of FIG. 17A.

FIG. 18 is a side elevation view of another alternative to the fifth embodiment.

FIGS. 19A and 19B schematically illustrate sections of molds that may be used to define pull element guides in the disclosed embodiments when formed using injection molding or metal molding processes.

FIG. 20 is a cross-section view of the segment of FIGS. 3A and 3B.

FIG. 21 shows a sixth embodiment of a shaft in a straight configuration;

FIG. 22 is an exploded view of two segments of the embodiment of FIG. 21;

FIG. 23 shows two segments of the embodiment of FIG. 21 in their nested configuration;

FIG. 24 is a plan view of a segment of the embodiment of FIG. 21;

FIG. 25 shows the shaft of FIG. 21 in an articulated position;

FIG. 26 is a cross-section view showing three segments of the shaft of FIG. 21 in an articulated position.

### DETAILED DESCRIPTION

The present application shows and describes shafts having sections that are deflectable or steerable through actuation of pull elements or other actuation components. The shafts may be incorporated into the designs of deflectable medical instruments. In the description that follows, the deflectable shafts are described as deflectable sections for instrument delivery tubes or ports of the type having a lumen through which other medical instruments are removably deployed during a procedure. The deflectable shaft sections allow the medical instruments to be supported and steered or deflected using actuation components of the shaft. A tubular liner of PTFE or other material may extend longitudinally through the lumen to form a smooth passageway for movement of instruments through the shaft. Medical instruments that may be used through such tubes include, but are not limited to, flexible-shaft forceps, graspers, dissectors, electrosurgical instruments, retractors, scopes, and tissue securing devices such as suture devices or staplers. A skin formed of a thin flexible membrane or material may cover the segments to prevent surrounding body tissue or other material from passing into the spaces between adjacent segments, or from being pinched or captured between adjacent segments. The skin is preferably loose enough that it will not resist deflection of the shaft when the pull elements are actuated.

Alternatively, the disclosed deflectable shafts may instead be incorporated into the designs of other instruments, such as surgical tools or scopes so that they can be deflected for or during use within the body. In embodiments of this type, an end effector (e.g. grasper, forceps, staple head, etc.) may be positioned at the distal end of the shaft for use in carrying out a procedure.

In certain of the disclosed embodiments, a deflectable shaft is formed of alternating segments, each of which has a first end or face contacting an adjacent segment along a first plane, and a second (opposite) end or face contacting an adjacent segment along a second plane that is orthogonal to the first plane. In some embodiments, the alternating segments are first and second segments having differently shaped contacting ends/faces. In other embodiment the alternating segments are identical to one another but are positioned such that segments having their first contacting end/face facing distally are alternated with segments having their second contacting ends/faces facing distally. In these embodiments, the first and second contacting ends/faces are shaped differently from one another.

A deflectable shaft using principles disclosed herein may comprise a portion of the full length of an instrument shaft. For example, the deflectable shaft may be positioned on a shaft that also includes a rigid shaft section having a fixed shape, a flexible shaft section (e.g. a flexible tube), or a rigidizable or “shape-lock” shaft section. In such embodiments, the deflectable shaft may be coupled to the distal end of the rigid, flexible, or rigidizable shaft section as described in U.S. application Ser. No. 12/846,788 (Attorney Docket No. TRX-2520), entitled DEFLECTABLE INSTRUMENT PORTS, filed Jul. 29, 2010. In other applications, the deflectable shaft section may be used as a proximal or intermediate portion of an instrument shaft. In still other applications, the deflectable shaft may extend the full length of an instrument shaft.

### First Embodiment

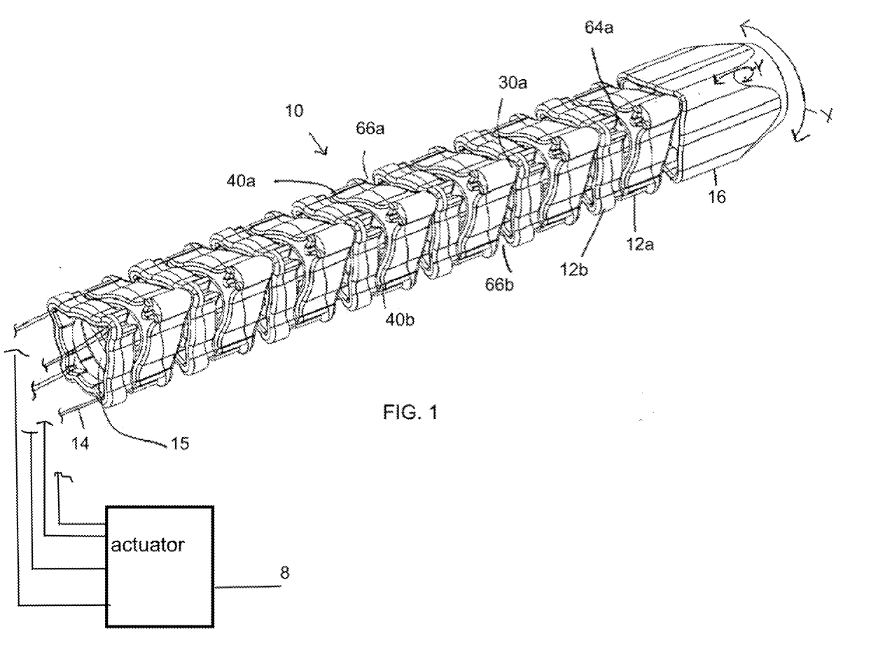

In a first embodiment shown in FIG. 1, a deflectable shaft section 10 is constructed using a plurality of segments 12a, 12b strung over a plurality of actuation elements 14, which may be wires, cables, filaments, ribbons, or other materials suitable for this purpose. In this description, the terms “pull elements” or “pull wires” may be used as short hand to refer to any of these types of actuation elements. In one embodiment, stainless steel wires are used. The pull elements are coupled to an actuator 8, shown schematically, which may be of the type shown and describe in the co-pending applications incorporated by reference herein, or which may take other forms known to those skilled in the art. In this and the other drawings, the areas of the pull elements that extend through and between the segments are not shown for purposes of clarity.

A distal tip 16 is coupled to the distal end of the shaft 10 and anchors the distal ends of the pull elements 14. The segments 12a, 12b and the distal tip 16 include central bores that are longitudinally aligned to form a lumen 15 in the shaft 10. The lumen 15 has a diameter sized to accommodate surgical instruments passed through the shaft for use in the body.

The segments 12a, 12b may be formed of rigid material such as nylon, glass-filled nylon, acetal, polycarbonate, glass-filled polycarbonate, stainless steel (which may be metal injection molded), or others. In other embodiments, the segments may be formed of stamped sheet metal. The first and second segments may be formed of the same materials or of different materials. For example, in one embodiment the first (longitudinally longer) segment 12a is formed of glass-filled Nylon while the second (longitudinally shorter) segment 12b is formed of stainless steel.

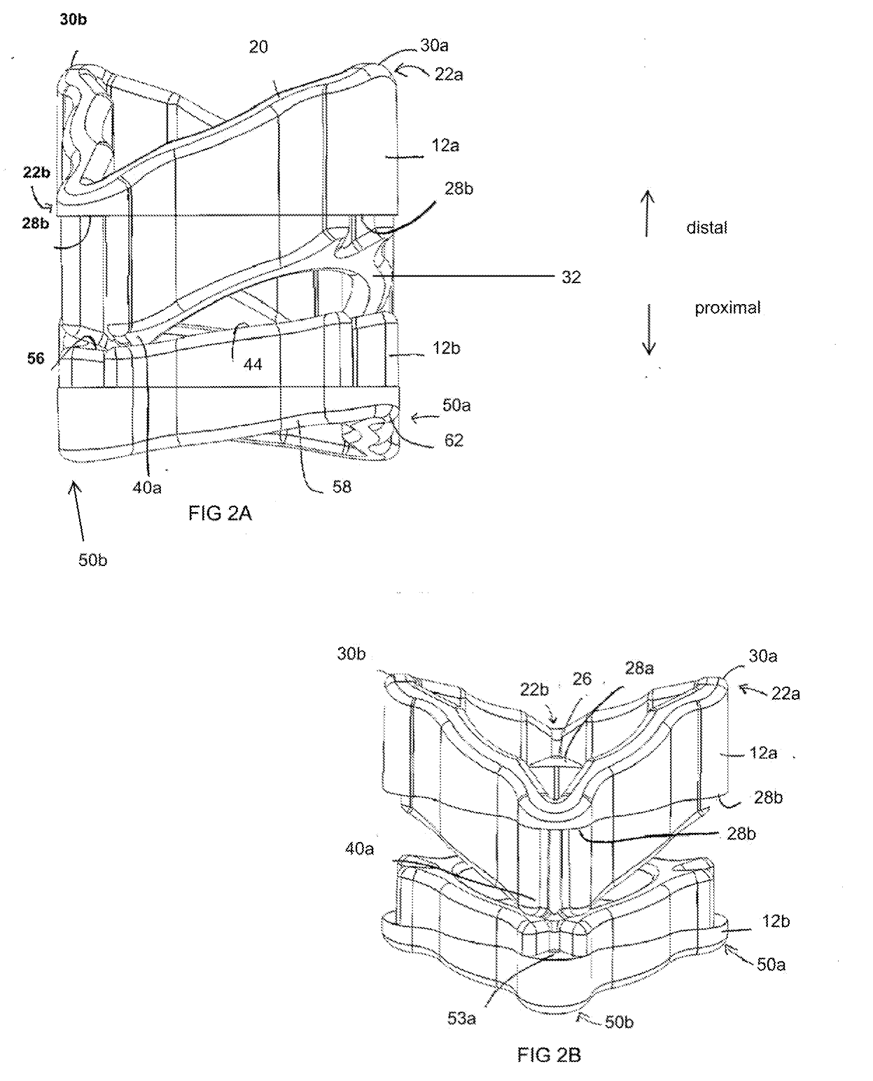

Segments 12a, 12b are constructed to form rocker joints, such that adjacent segments can rock relative to one another in response to application of tension on the pull elements. Note that adjacent segments 12a, 12b are in contact with one another but preferably do not have a direct physical connection to one another by hinges, rivets or other means. In the first embodiments, the segments comprise first segments 12a alternating with second segments 12b along the length of the deflectable shaft section 10. FIGS. 2A and 2B illustrate one first segment 12a and one second segment 12b. Notations of “distal” and “proximal” on this figure and others in this description are included for purposes of convenience and should not be construed to limit the orientation of the segments in practice.

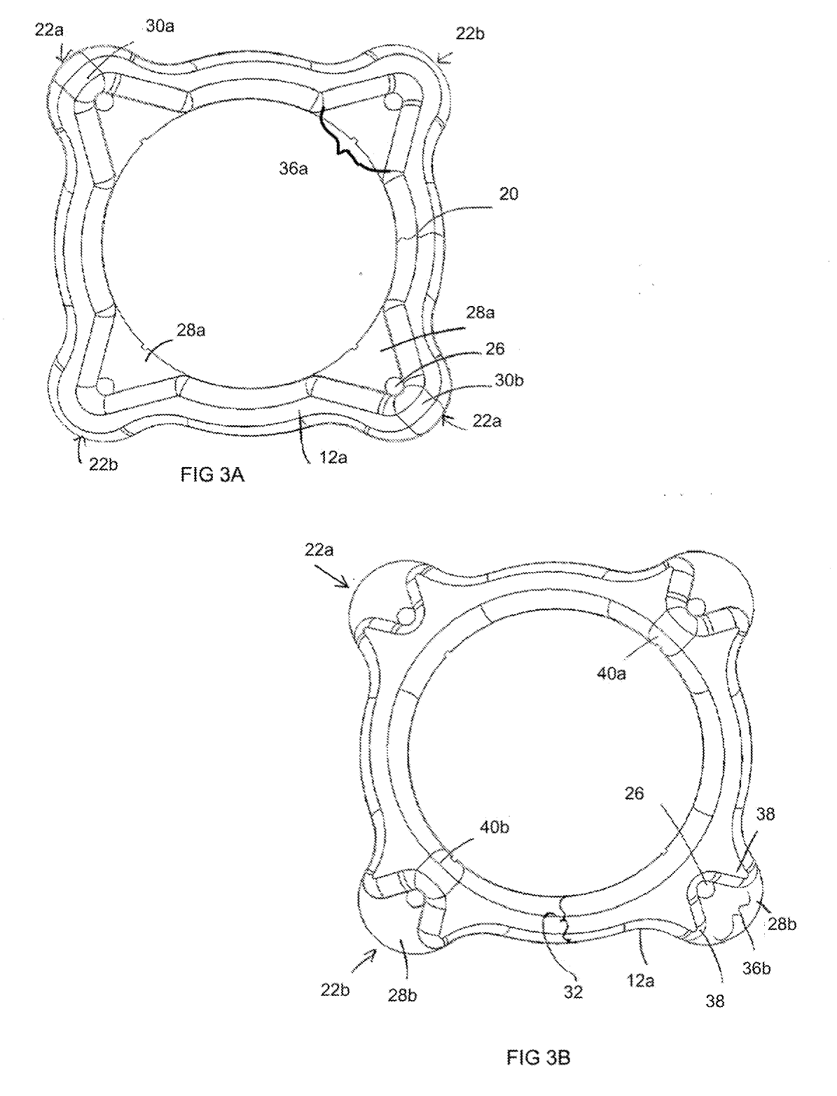

As shown in the distal plan view of FIG. 3A, the first segment 12a has an outer profile that is generally square with rounded corner sections 22a, b. Contoured sides are disposed between the corner sections 22a, b. The distal end of the first segment includes a distal face 20. This face, as well as the others defined below, may have a planar or non-planar surface. The distal face 20 is the distal facing surface of a wall 20a having an outer surface that defines the generally square perimeter of the segment 12a, and an inner surface that (at the corner sections 22a, 22b) defines longitudinal channels 36a, and that (between the corner sections 22a, 22b) is longitudinally aligned with the central bore 15a.

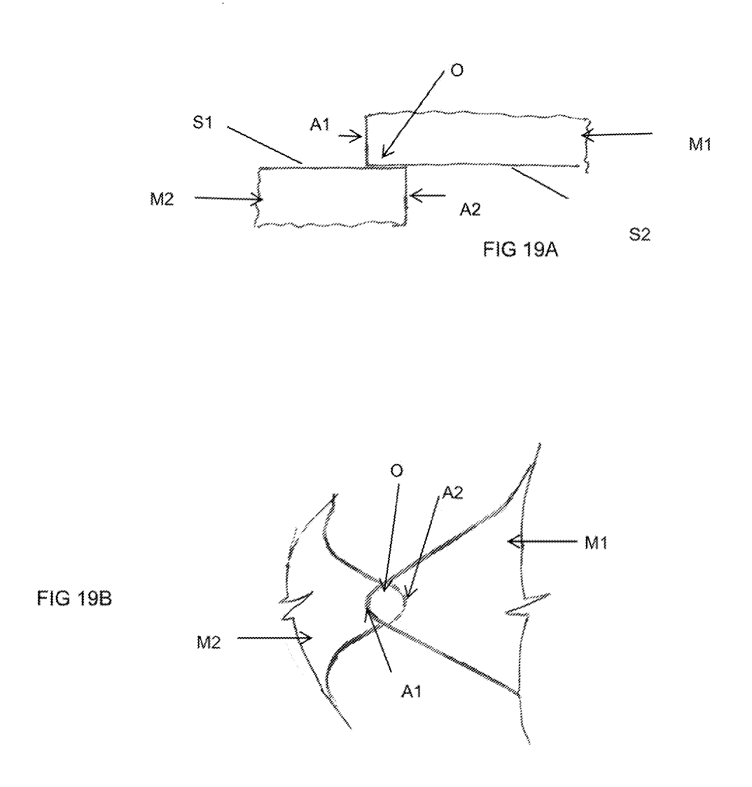

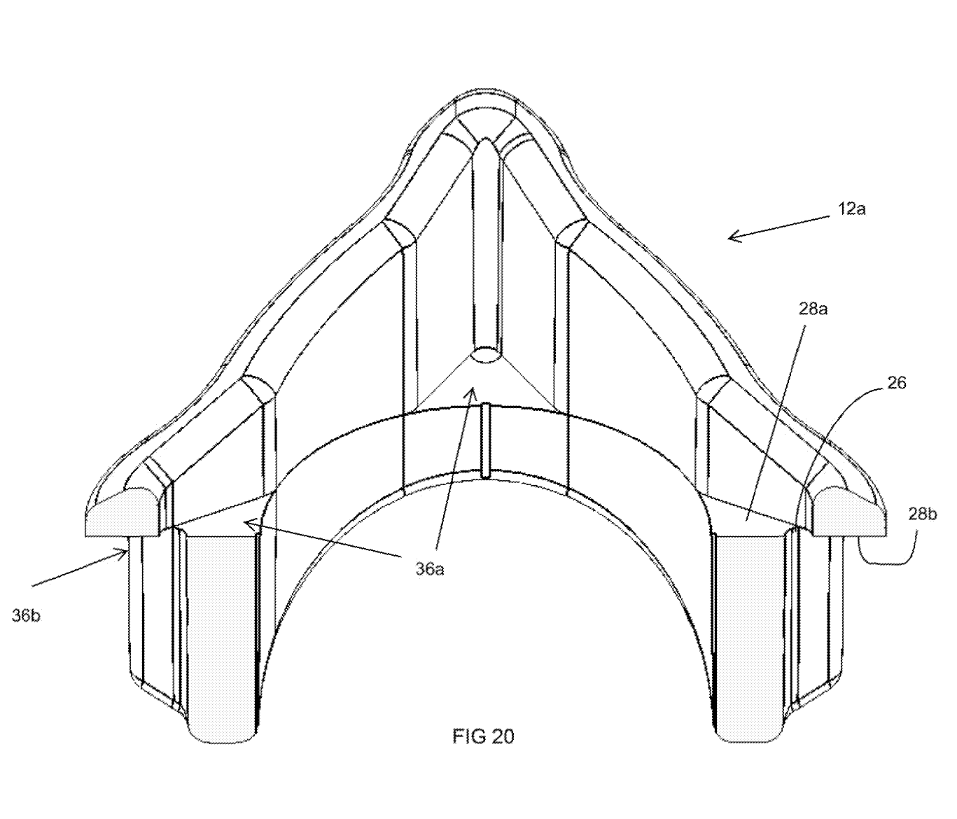

Guides 26 for receiving the pull elements (not shown) are located at the corner sections 22a, 22b. In the illustrated embodiment, the guides 26 are bounded by the edges of opposed, preferably planar, floor members 28a,b disposed within the corner sections 22a, 22b. See also FIG. 20. In some embodiments the guides 26 may be longitudinal holes or bores formed in the segments. However, conventional hole formation in the injection molding process typically uses pins to define holes that are needed in molded components. This process can be unsuitable for forming holes having the small diameters that may be desired for the guides 26 (e.g. where guides 15/1000″ in diameter are desired for use with actuation elements that are 14/1000″ diameter). For this reason, the guides 26 are formed by using a unique molding process, described below in connection with FIGS. 19A through 20, that allows formation of guides as bounded openings through the segments, without the use of pins. This method allows the segments to be easily and economically manufactured via injection molding and metal injection molding processes.

The wall 20a extends around the guides 26, defining the four generally v-shaped or wedge-shaped channels 36a longitudinally aligned with the guides 26. See also FIG. 20.

As shown in the plan view of FIG. 3B, the proximal end of the first segment 12a includes a proximal face 32. The proximal face is the proximally-facing surface of a wall 32a having an inner surface that defines the bore 15a. At the corner sections 22a, 22b, the outer surface of the wall 32a curves inwardly and then outwardly to expose the guides 26 and to define four generally v-shaped or wedge-shaped channels 36b (e.g. between adjacent protrusions 38 as shown) longitudinally aligned with the guides 26. See also FIG. 20. Between the corner sections 22a, 22b, the outer surface of the wall 32a is longitudinally aligned with the outer surface of the distal end wall 20a

As best seen in FIGS. 2A and 2B, the distal face 20 of the first segment 12a slopes in a proximal to distal direction from the corner sections 22b to the corner sections 22a, defining distally-extending peaks 30a, b at the corner sections 22a. The proximal face 32 on the first segment 12a similarly slopes in a distal to proximal direction from the corner sections 22a to the corner sections 22b to define proximally-extending peaks 40a, b at the corner sections 22b. When viewed longitudinally, the distal-most points of the distally-extending peaks 30a, b define a first longitudinal plane and the proximal-most points of the proximally-extending peaks 40a, b define a second longitudinal plane, with these planes being transverse to one another. In this embodiment, since the peaks 30a, 30b, 40a, 40b are at the corner sections, the distally extending peaks 30a, b are offset ninety degrees from the proximally extending peaks 40a, b when viewed longitudinally, the first and second longitudinal planes are orthogonal to one another.

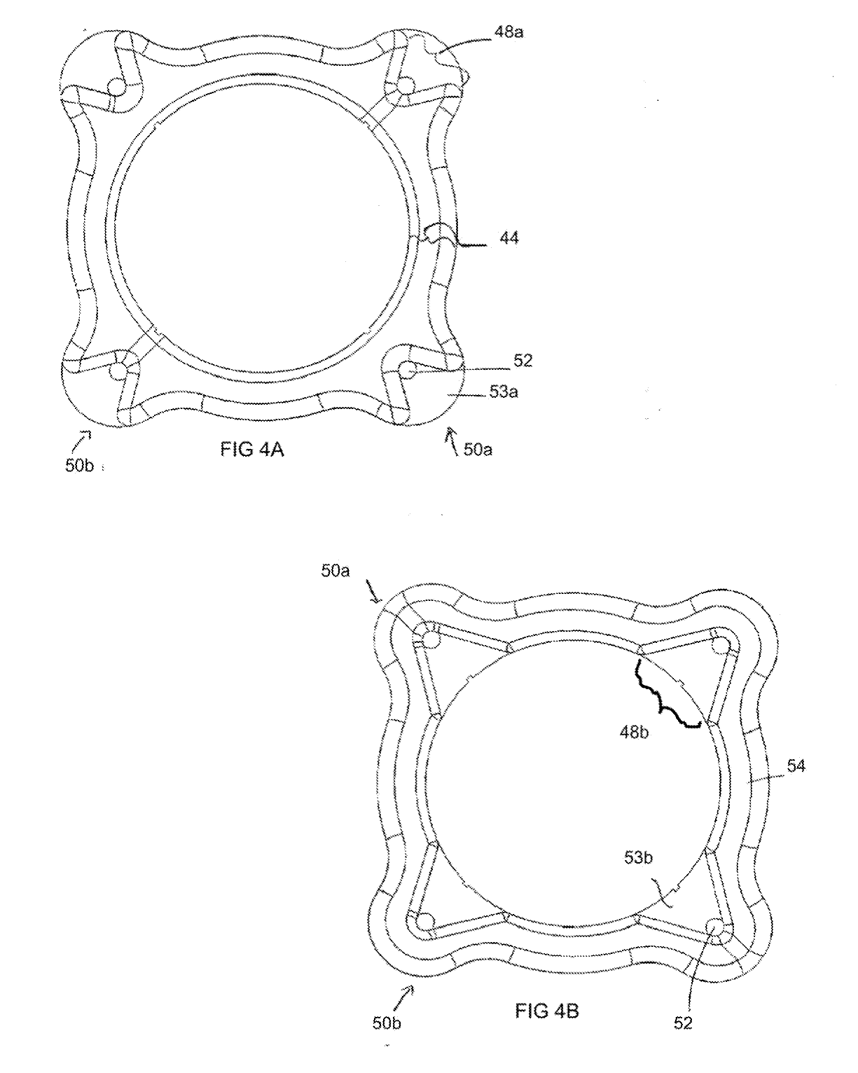

The second segment 12b includes rounded corner sections 50a, 50b and in preferred embodiments has an outer footprint size and other features similar or identical to those of the first segment 12a. As shown in the plan view of FIG. 4A, the second segment's distal end has a distal face 44 on a wall 44a that is similar to the wall 32a of the first segment 12a in that it curves inwardly and then outwardly to define generally v-shaped channels 48a. The proximal end of the second segment 12b, shown in plan view in FIG. 4B, has a wall 58a shaped similarly to the wall 20a at distal face 20 of the first segment 12a and defines generally v-shaped channels 48b. Pull element guides 52 are positioned in the corner sections 50a, b (e.g. in planar or non-planar floors 53), and are longitudinally aligned with the apexes of the channels 48a, 48b. Contoured edges 54 extend between the corner sections 50a, 50b.

As best seen in FIG. 2A, the distal face 44 of the second segment 12b slopes in a distal to proximal direction from the corner sections 50a towards the corner sections 50b to form generally v-shaped saddles 56. The proximal face 58 of the second segment similarly slopes in a proximal to distal direction from the corner sections 50b towards the corner sections 50a to form generally v-shaped saddles 62. As with the peaks of the first segment, the proximal and distal saddles of the second segment are offset from one another, and in the illustrated embodiment they are offset by ninety degrees, thus defining longitudinal planes that are orthogonal to one another.

Referring again to FIG. 1, the first and second segments 12a, 12b are arranged such that when the shaft 10 is in its straight orientation, the peaks of the first segments are seated against the corresponding saddles of the adjacent second segments. Thus, for a given first segment 12a, the distal peaks 30a, b of the first segment 12a are seated against the proximal saddles 62 of the distally-adjacent second segment 12b, and the proximal peaks 40a, b of the first segment 12a are seated against the distal saddles 56 of the proximally-adjacent second segment 12b. Given the orientations of the peaks and saddles on the first and second members, respectively, when the shaft 10 is in the straight orientation, the first segments contact their distally adjacent second segments at contact positions in a first longitudinally-extending plane and they contact their proximally adjacent second segments at contact points in a second longitudinally-extending plane that is perpendicular to the first longitudinally-extending plane.

In this embodiment, the angles of the peaks of the first segment 12a are steeper than those of the saddles of the second segment 12b, and the longitudinal length of the first segment is larger than that of the second. When the segments 12a, 12b are assembled to form a shaft, the pull elements 14 (FIG. 1) are threaded through the guides 26, 52 in the segments and anchored at the distal tip of the shaft. The pull elements 14 are laterally restrained by the v-shaped channels 36a, b and 48a, b.

Given the sloped distal and proximal ends or faces of the segment walls, this arrangement leaves first gaps 64a, b and second gaps 66a, b between the segments 12a, 12b. The first gaps 64a, b (gaps 64b not visible in FIG. 1) are disposed between each second segment 12b and its distally-adjacent first segment 12a. These first gaps 64a, b are longitudinally aligned with the corresponding set of distally-extending peaks 30a, b (peaks 30b not visible in FIG. 1) of the first segments 12a. The second gaps 66a, b are disposed between each second segment 12b and its proximally-adjacent first segment 12a. These second gaps 66a, b are longitudinally aligned with the corresponding set of proximally-extending peaks 40a, b of the first segments 12a.

Tensioning the pull elements 14 in a manner that closes the first gaps 64a or the first gaps 64b causes deflection of the shaft in direction Y indicated by arrow Y (into and out of the page) in FIG. 1. Tensioning the pull elements 14 in a manner that closes the second gaps 66a or 66b causes deflection of the shaft in direction X indicated by arrow X (side to side in the view of FIG. 1). This arrangement allows for full 360° deflection of the shaft 10 using simultaneous tensioning of various combinations of the pull elements to varying degrees.

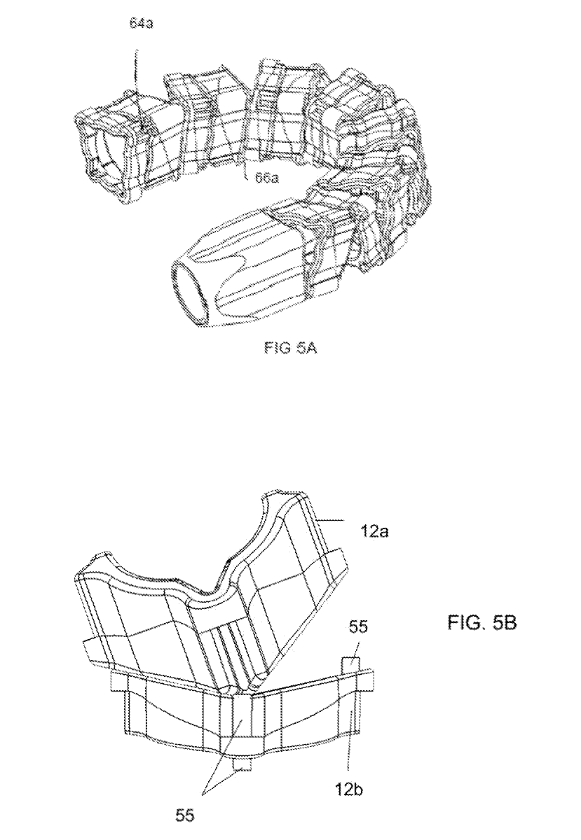

FIG. 5A shows the shaft 10 after it has been fully deflected into one bent configuration. As can be seen, in this arrangement first gaps 64a and second gaps 66b are both closed along the inner edge of the formed curve, bringing the adjacent distal and proximal faces of the segments' walls into contact with one another along that edge. FIG. 5B is a close-up view of two of the segments shown in FIG. 5A in a deflected configuration. In this figure, optional tubular liners 55 extend through the pull element guides 52 of segment 12b, so as to reduce friction between the pull elements and the segment material surrounding the guides. Reduction of friction may be particularly desirable where both the pull elements are the segments are formed of stainless steel or other materials that will generate undesirable levels of friction. Lower levels of friction are desired to minimize the amount of force the user must apply to the actuators to deflect the shaft. Liners 55 are preferably made of PTFE or other suitable polymer or other material that will cause the desired reduction in friction.

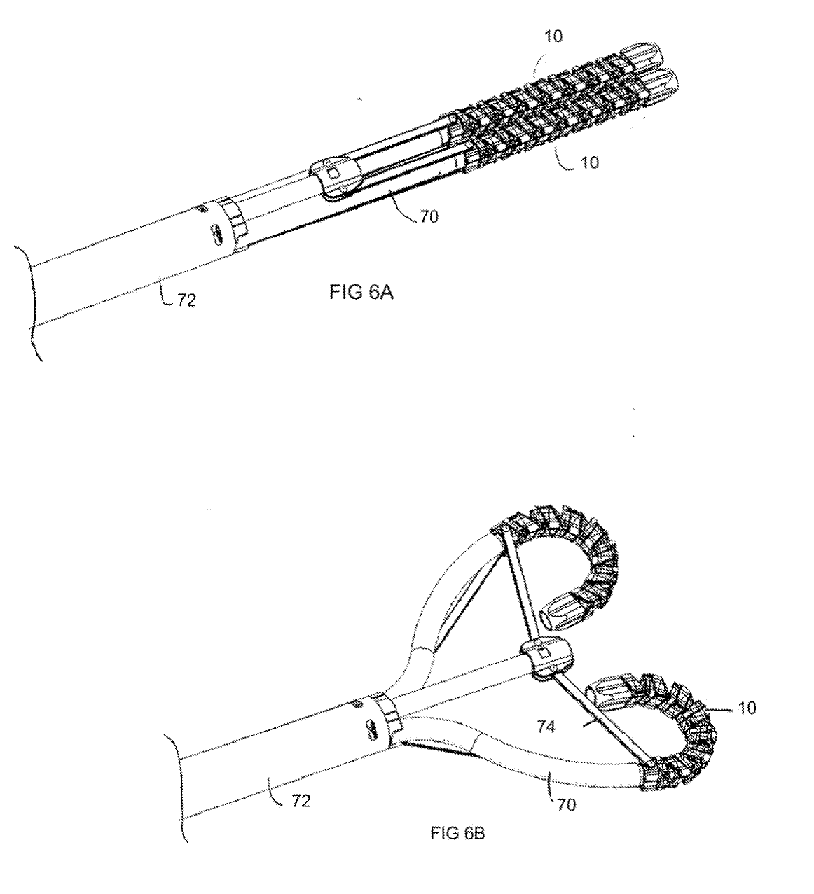

FIGS. 6A and 6B illustrate the use of shafts 10 as part of an instrument access system 80 of the type disclosed in U.S. application Ser. No. 12/639,307, filed Dec. 28, 2009, which is hereby incorporated herein by reference. Here the shafts 10 form the distal ends of instrument delivery tubes 70 that extend through an outer tube 72. The pull elements (not shown in FIGS. 6A and 6B) extend through the instrument delivery tubes and are coupled to actuators (not shown) that are manipulated by a user to tension the pull elements for deflection of the shaft 10. The actuators may be of the type shown and described in the prior application or they may have alternative designs.

The portions of the instrument delivery tubes 70 that are proximal to the shafts 10 may have segmented construction similar to that of the shafts 10, or they may be formed of extruded tubing or other material. Links 74 are used to separate the shafts 10 after the distal end of the system 80 has been introduced into a body cavity as described in the prior application. The pull elements are then manipulated to deflect the shafts 10 into bent positions such as those shown in FIG. 6B.

It should be noted that while the system shown in FIGS. 6A and 6B is given as an example of systems into which deflectable instrument delivery tubes using the shafts 10 may be used, similar instrument delivery tubes may also be used with any other type of access system, laparoscopic port, trocar, cannula, seal, catheter, introducer, etc. suitable for use in giving access to a body cavity.

### Second Embodiment

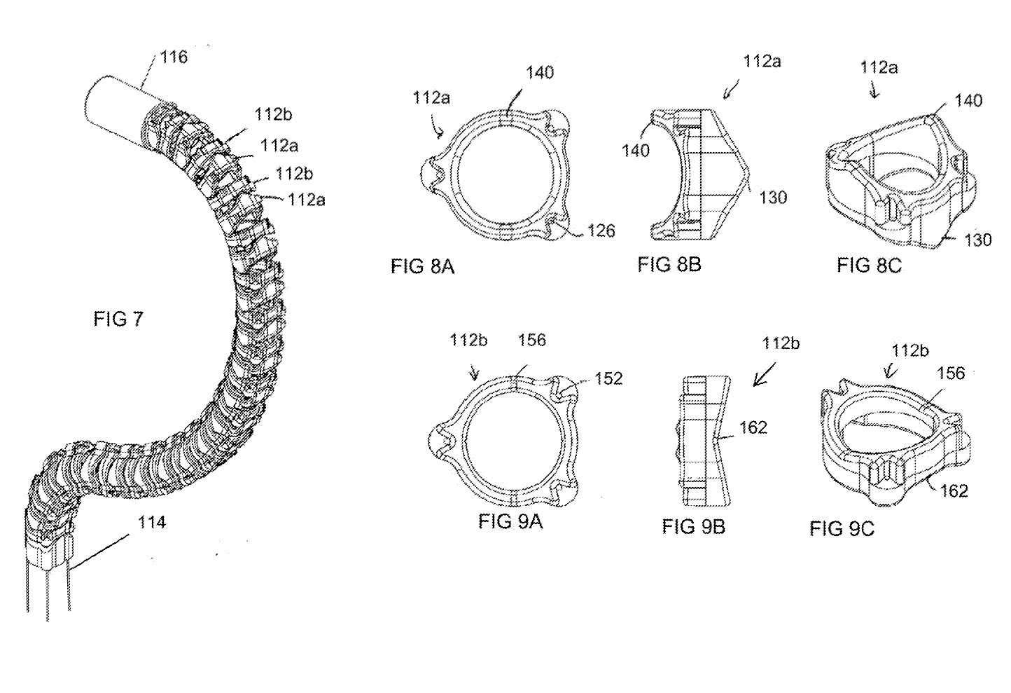

FIG. 7 shows a second embodiment of a shaft 110 deflected to a bent position. The FIG. 7 embodiment is similar to the FIG. 1 embodiment, but is modified to use three rather than four pullwires. As with the first embodiment, the second embodiment utilizes first segments 112a alternating with second segments 112b strung over pull elements 114 along the length of the deflectable shaft section 110.

Referring to FIGS. 8C and 9C, first segments 112a are formed to have a pair of distally-extending peaks 130 which seat against corresponding saddles 156 on the distal end of corresponding second segments 112b. Each first segment 112a additionally includes a pair of proximally-extending peaks 140 which seat against corresponding saddles 162 on the proximal end of the corresponding second segment 112b. Guides 126 and 152 are provided for receiving the pull elements. As with the first embodiment, up to 360° deflection of the shaft 110 can be achieved through manipulation of the pull elements to cause x- and y-movements of the segments to close gaps between various portions of their distal and proximal faces.

### Third Embodiment

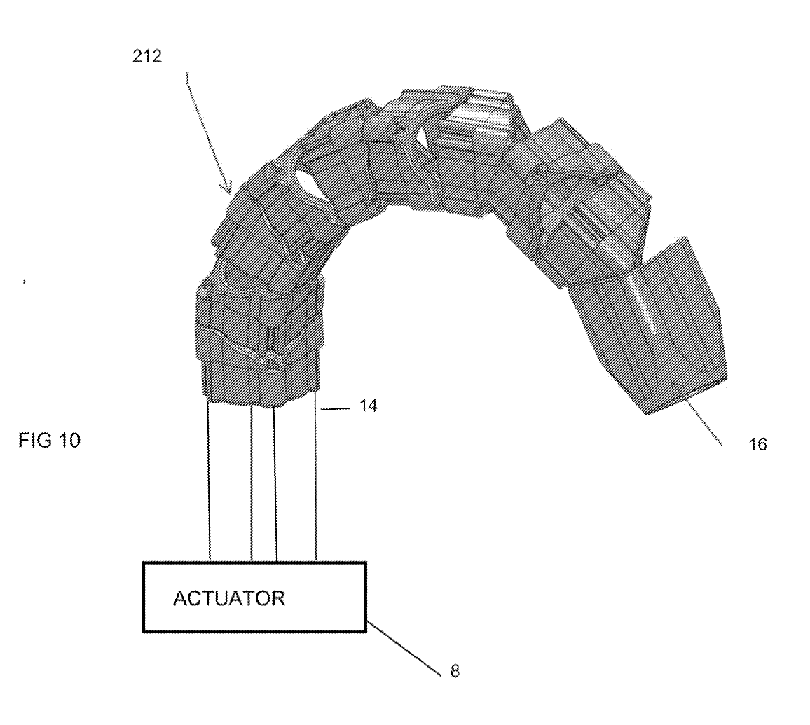

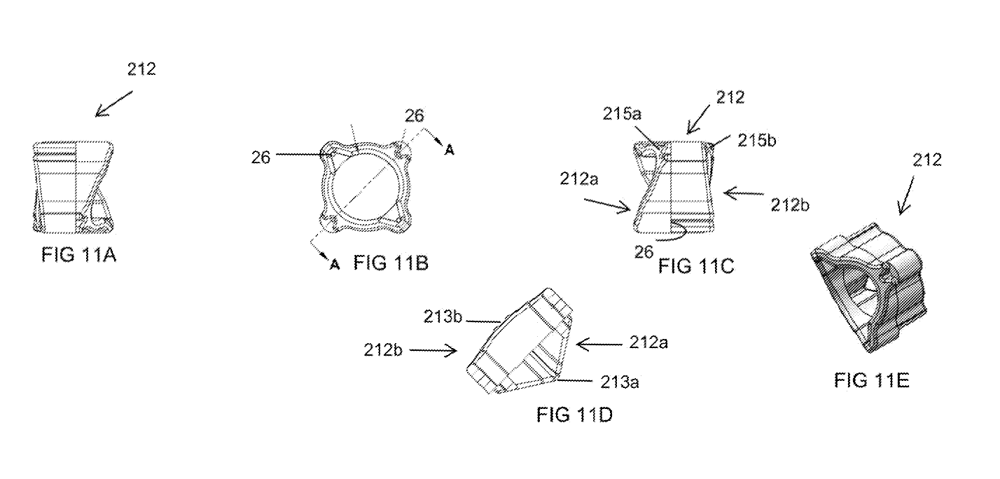

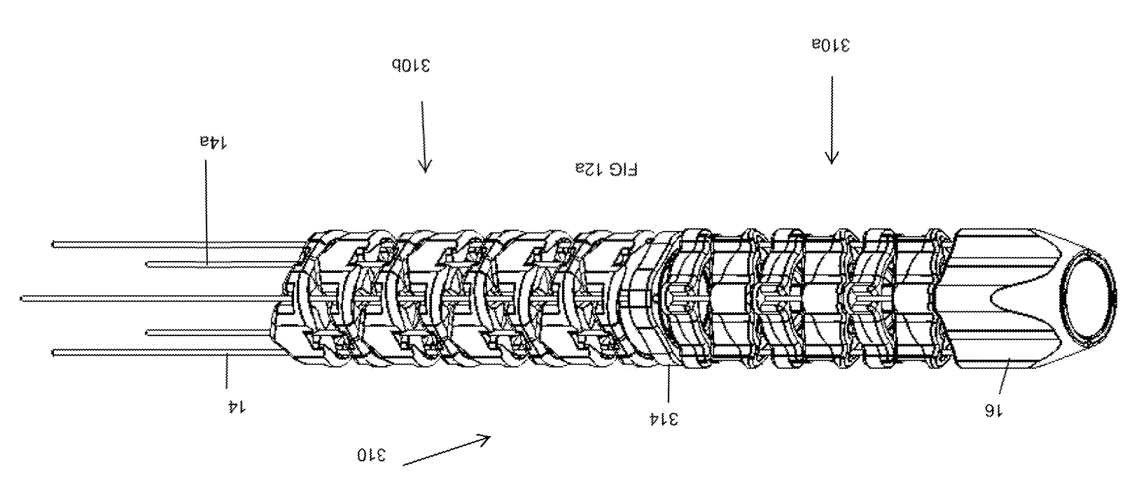

FIG. 10 shows a third embodiment of a shaft 212 deflected to a bent position. The FIG. 10 embodiment is similar to the FIG. 1 embodiment, but is modified to use a single type of segment 212, shown in various views in FIGS. 11A through 11E, rather than using different first and second segments. The segment 212 includes a first face 212a which is similar to one of the faces (distal or proximal) of the first segment 12a of the first embodiment, and a second face 212b which is on the end of the segment opposite from the first face and which is similar to one of the faces (distal or proximal) of the second segment 12b of the first embodiment. As with the first and second segments 12a, 12b of the first embodiment, the first face and the second face each includes a peak 90 degrees offset from a saddle. The peaks 213a of the first face 212a are longitudinally aligned with the peaks 213b of the second face 212a and the saddles 215a, b are likewise aligned. On the first face, the peaks and saddles extend at a larger angle than do the peaks and saddles on the second face. The orientations of the segments are alternated, such that a first one of the segments will have its first face 212a facing distally, while its proximal and distal neighbors will have their second faces 212b facing distally. This forms rocker joints between the segments as shown in FIG. 10 and in a manner similar to that described with respect to the first embodiment.

### Fourth Embodiment

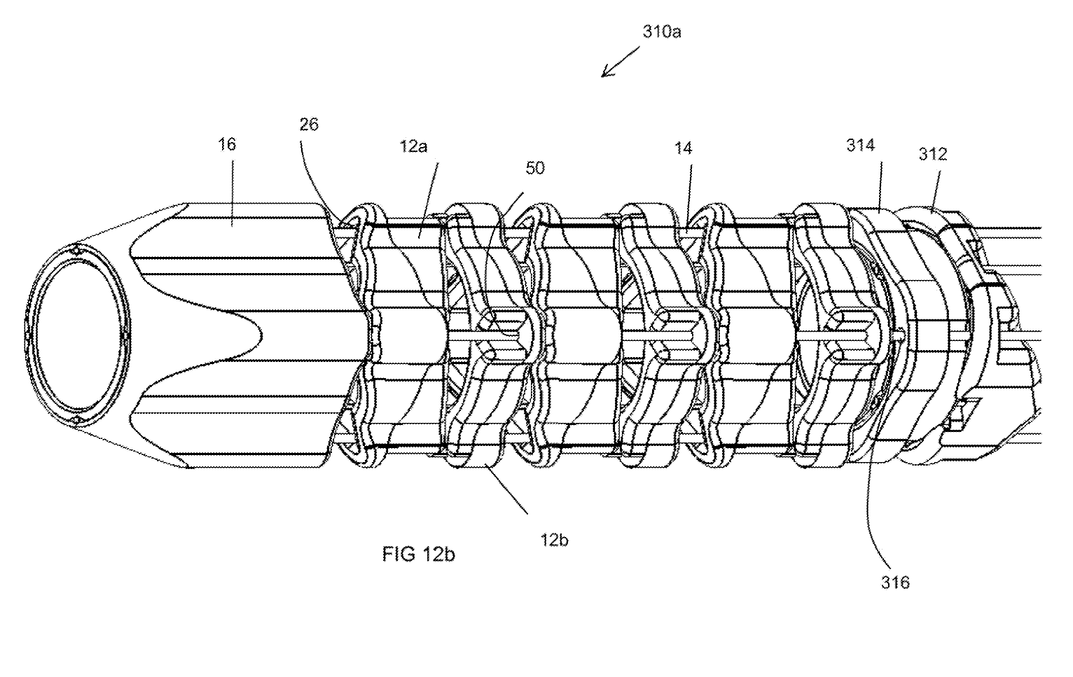

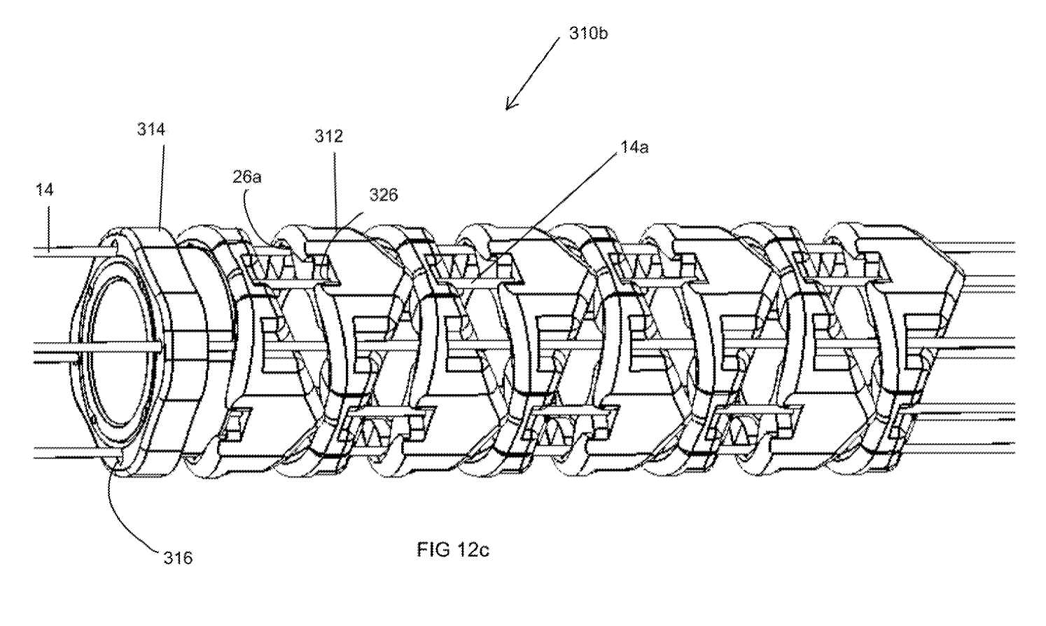

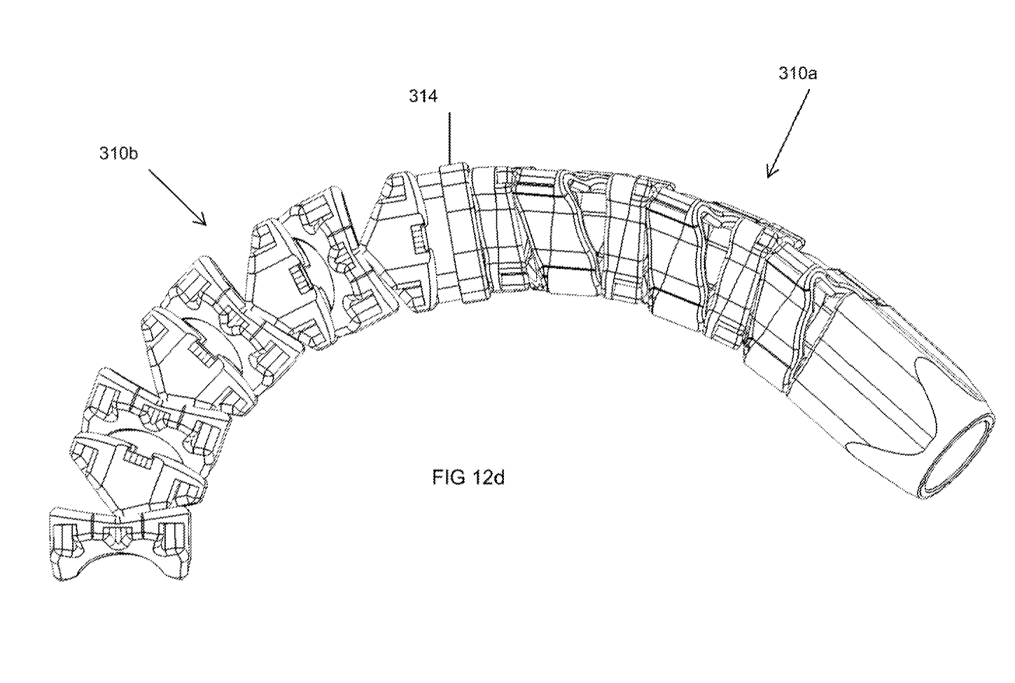

FIG. 12a shows a fourth embodiment of a deflectable shaft 310, which includes a distal section 310a and a proximal section 310b, each of which is controlled by its own dedicated set of actuation elements. This modification allows the loads associated with each separate section 310a, 310b to be resolved over a shorter distance than would be the case if a single set of actuation elements controlled deflection of the combined length of sections 310a and 310b.

Enlarged views of the first and second sections are shown in FIGS. 12b and 12c, respectively. First section 310a comprises segments 12a, 12b of the type described with respect to the third embodiment. The second, more proximal, section 310b comprises segments 312 similar to the segments 12a, with guides 26a similar to guides 26a of segment 12a. Segments 312 also include four additional guides 326 that are offset from the guides 26a by an angle of 45 degrees. Note that the proximal section 310b is oriented such that the distally and proximally extending peaks of the segments 312 are offset 45 degrees from the corresponding peaks of the segments of the segments 12a, and such that the guides 26, 50 of the distal section segments 12a, 12b are longitudinally aligned with the guides 326 of the proximal section segments 312. An intermediate segment 314 is positioned between the distal and proximal sections 310a, 310b, and includes guides 316 longitudinally aligned with the guides 326 of the proximal section 312a and guides 26, 50 of the distal section 310a.

When the fourth embodiment is assembled, a first set of four actuation elements 14 extends through guides 326 in the proximal section 310b, guides 316 in the intermediate segment 314, and guides 26, 50 in the distal section. These actuation elements 14 are anchored at the distal end of the distal section 310a, such as at the most distal segment 212 or at the distal tip 16. Manipulation of these actuation elements controls bending of the distal section 310a as described with prior embodiments.

A second set of four actuation elements 14a extends through guides 26a in the proximal section 310b. These actuation elements are anchored at the distal end of the proximal section, such as at the distal-most segment 312 or at the intermediate segment 314. Manipulation of these actuation elements controls bending of the proximal section 310b. The proximal ends of the actuation elements 14, 14a are coupled to one or more actuators 318, which may be of a type that engages the pull elements in accordance with movement of the handle of an instrument passed through the shaft 310 as disclosed in the previously incorporated applications. Such an actuator might be an actuation system comprised of two separate actuators, one that actuates elements 12 and another that actuates elements 14a. FIG. 12d, in which the actuation elements are not shown, shows the fourth embodiment in the deflected position.

### Fifth Embodiment

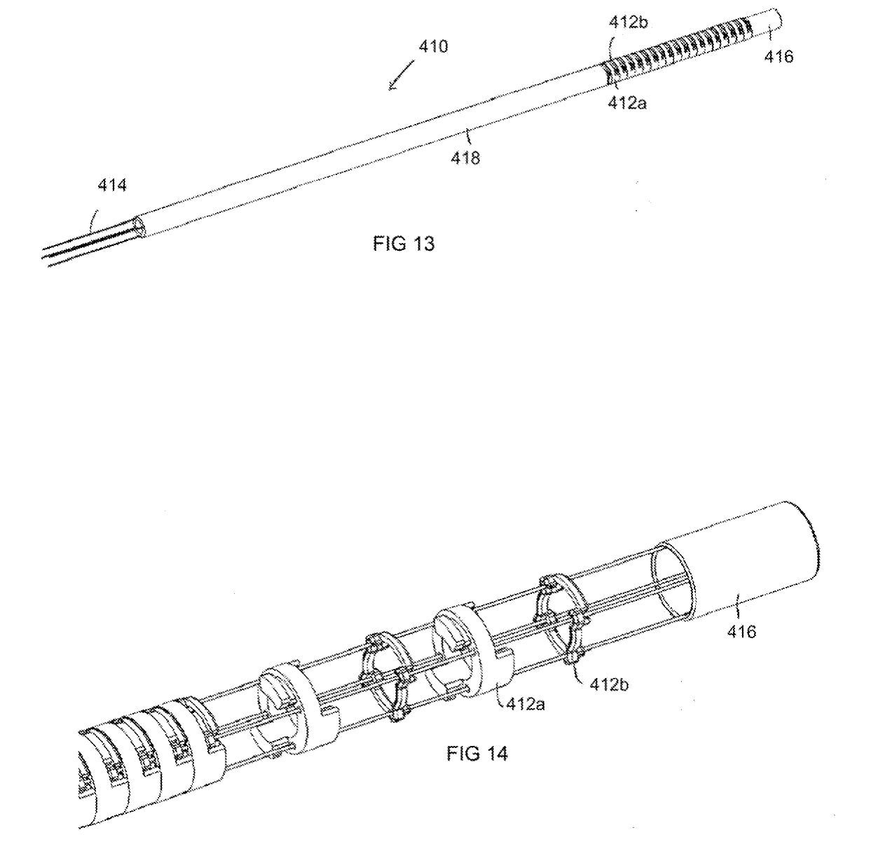

A fifth embodiment of a deflectable shaft 410 is shown in FIG. 13. In this embodiment, the deflectable shaft formed of alternating compressible and rigid segments

Shaft 410 includes a plurality of segments 412a, 412b strung over a plurality of pull elements 414. The pull elements 414 are anchored by a tubular tip 416 at the distal end of the shaft 410. The shaft 410 may include a proximal portion 418 formed of an elongate section of tubing.

FIG. 14 is an exploded view of the distal end of the shaft 410. The segments forming the shaft comprise compressible segments 412a and rigid segments 412b. The compressible segments 412a may be formed of compressible material such polyisoprene, silicone, or other suitable material, while the rigid segments 412b may be formed of rigid material such as nylon, glass-filled nylon, acetal, polycarbonate, glass-filled polycarbonate, stainless steel (which may be metal injection molded), or others. The compressible material of the segments 412a gives the shaft sufficient flexibility to allow the desired degree of deflection while minimizing the amount of tension needed to be placed on the pull elements in order to accomplish bending. The rigid material of the segments 412b helps to prevent the shaft from buckling during use.

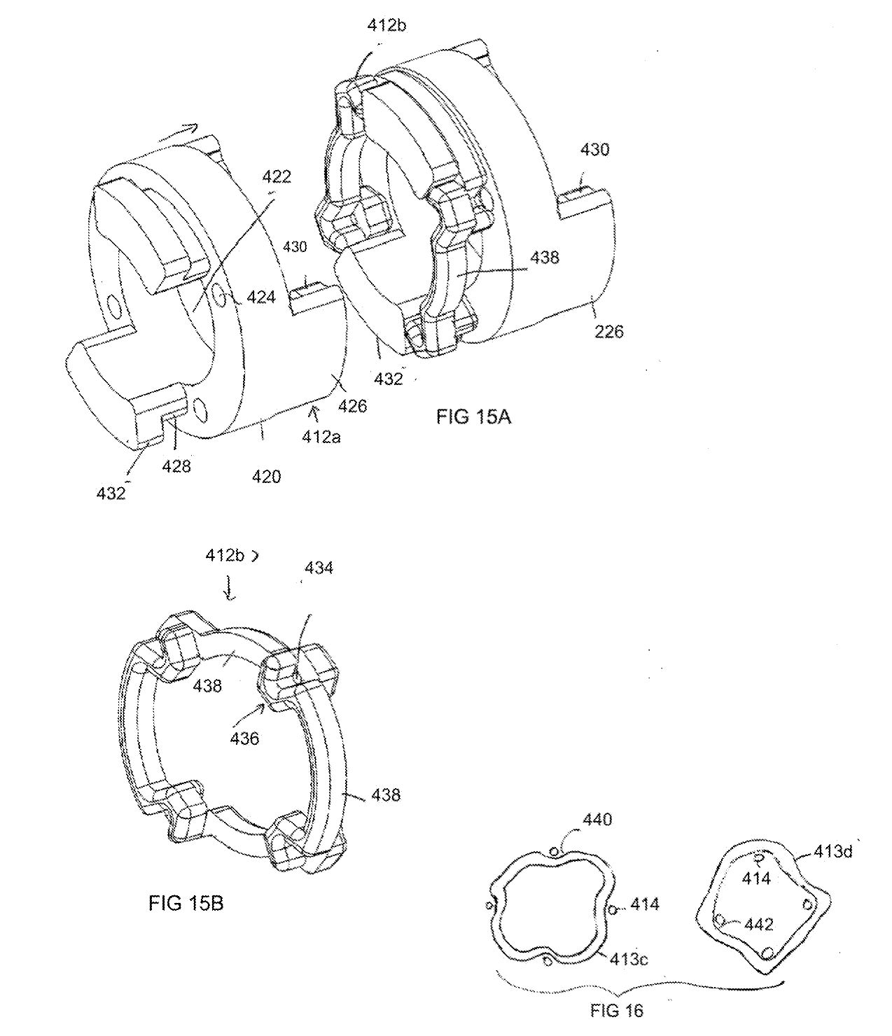

The segments 412a, 412b may be fabricated to have any of a variety of shapes and features. FIG. 15A shows one design for the segments 412a which incorporates features for interlocking the segments 412a, 412b. According to this example, compressible segment 412a has an annular base 420 with a central opening 422. Four pull element guides 424 extend in a longitudinal direction through the base 220 and are spaced at 90 degree intervals. Two first members 426 extend longitudinally from one face of the base 420 on opposite sides of the central opening 422. On the opposite face of the base 420, a pair of second members 428 extends longitudinally from the base 420 on opposite sides of the central opening 422. The second members 428 are inwardly spaced from the outer edge of the base 420. Each first member has a lip 430 that extends radially inwardly as shown, and each second member 428 has a lip 432 that extends radially outwardly.

Referring to FIG. 15B, the rigid segments 412b are annular rings having pull element guides 434 and guides 436 spaced at 90 degree intervals to divide the rings into four equal arcs 438.

FIG. 15A illustrates the manner in which a rigid segment is assembled with the two adjacent compressible segments. As shown, on one side of the rigid segment 412b, two opposite arcs 438 of the rigid segments 412b are passed over and captured beneath opposite lips 432 of a compressible segment 412a. On the opposite side of the rigid segment 412b, the remaining arcs 438 are inserted beneath lips 430 of a second compressible segment 412a. Additional rigid segments and compressible segments are added in alternating fashion to form the shaft 410.

Although the segments 412a, 412b are designed with interlocking features, alternative embodiments may be provided without interlocking features. Moreover, the segments may be provided without guides for the pull elements. For example, alternative segment types 412c, 412d shown in FIG. 16 are provided without any such guides. Instead, segment types 412c and 412d are alternated to form the deflectable shaft, and the pull elements are woven between the segments such that they pass over the outer edge of the segment 413c and along the inner edge of the segment 413d as shown. The segments may be shaped to include guides 442, 444 on their inner or outer surfaces to aid in containing the pull elements. One of the segments 412c, 412d may be compressible while the other is rigid as in the previous embodiment, or both may be either compressible or rigid.

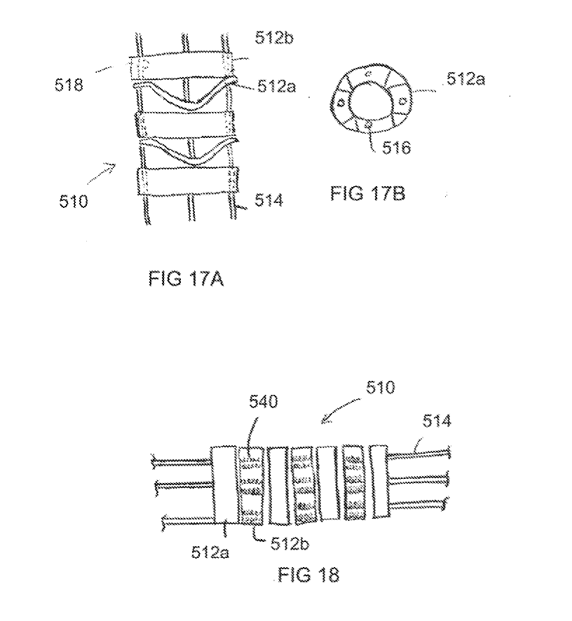

In another alternative to the fifth embodiment shown in FIG. 17A, the shaft 510 is formed of compressible segments 512a and rigid segments 512b, but in this case the compressible segments 512a are formed of annular wave springs as shown in FIG. 17B. The pull elements 514 extend through guides 516 in the springs and through corresponding guides 518 through the rigid segments 512b.

FIG. 18 shows yet another alternative to the fifth embodiment, in which both types of segments 512a, 512b are formed of compressible material such as silicone. However in this embodiment, the segments 512b have increased resistance to compression due to the presence of coil-pipe sections 520 embedded within the compressible material.

### Sixth Embodiment

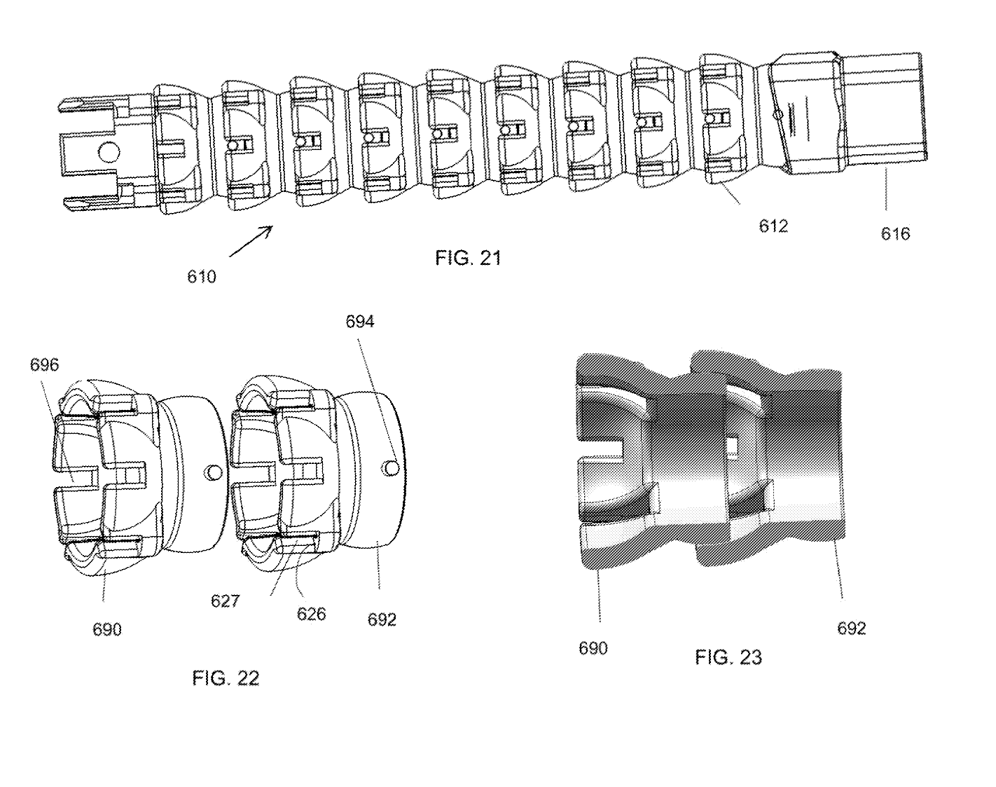

FIGS. 21 through 25 show a sixth embodiment of a shaft 610 in which each of the plurality of segments 612 may be identical to the others. The segments may be formed using materials and techniques similar to those described with respect to other embodiments.

Referring to FIG. 22, each segment includes a first portion 690 defining a socket having a concave (preferably partially-spherical) internal surface, and a second portion 692 having a convex (preferably partially-spherical) external surface. A plurality of the segments 612 are strung over pull elements as described in previous embodiments, with the pull elements engaged in the tip element 617 or in one of the more distal segments.

The segments are arranged such that the second portion 692 of one segment is disposed within the first portion 690 of an adjacent segment—with the partially-spherically surfaces in contact with one other. In the illustrated embodiment, the first portions are oriented proximally and the second portions are oriented distally. During use, actuation of the pull elements causes bending of the shaft 610, with adjacent segments articulating relative to one another with their adjacent partially-spherical surfaces in contact with one another. This ball and socket type arrangement allows full 360 degree articulation of each segment relative to its neighboring segment—thus allowing for smoother movement of the segments relative to one another. Moreover, as best shown in FIG. 23, this nested arrangement of segments forms a generally smooth interior lumen even when the shaft is articulated. The smooth interior lumen facilitates passage of medical instruments through it during use.

The segments 612 include anti-rotation features to prevent the segments from axially rotating relative to one another. As one example, anti-rotation members on one segment are engaged by anti-rotation features on the adjacent segments. In the drawings, the second portions 692 have anti-rotation posts 694 that are received in corresponding slots 696 formed in the first portions 690.

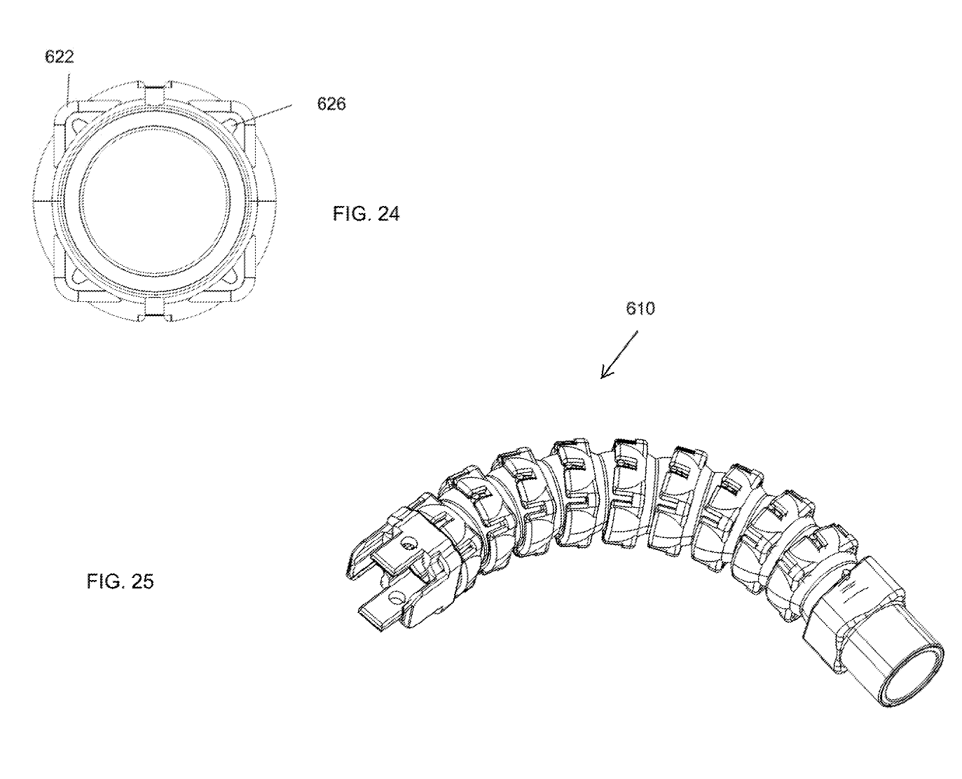

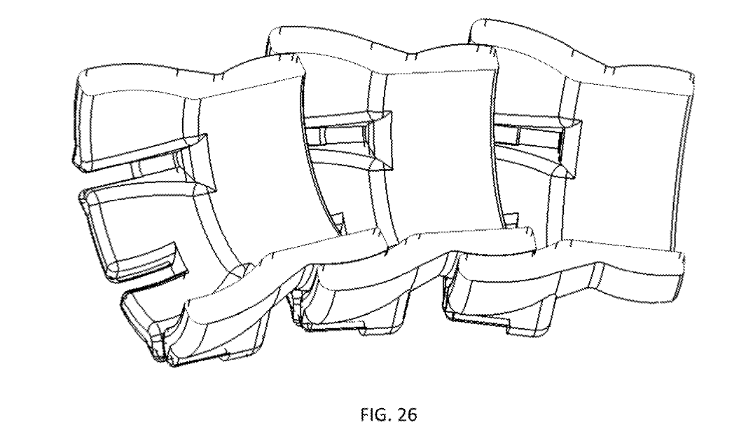

As best seen in FIG. 24, the segments include guides 626 for receiving the pull elements. The guides 626 are located at the first portion of each segment, and may extend through the wall of the first portion—e.g. as lumen extending through the wall. Alternatively, the guides 626 may be similar to the guides of the previous embodiments. For example, the first portion may have rounded corner sections 622 extending radially outwardly from the spherically-contoured outer-surface of the first portion. The guides 626 are located in these corner sections as shown. Longitudinal slots 627 in the spherically-contoured outer-surface of the first portion may be longitudinally aligned with the guides 626. If the segments are formed using molding techniques, the guides 626 may be formed using the technique described below.

Molding Process for Segments

The segments (e.g. segments 12a, 12b of FIG. 1) utilized in the various embodiments may be formed using a unique molding process that allows formation of guides for the actuation elements (see guides 26 in FIG. 3A) without the use of pins. While conventional molding techniques use pins to create molded pieces that include holes, the very small size of the guides 26 would require pins of such small diameter that the pins would either be too flexible to resist bending during molding, or made from materials that are prohibitively expensive for use in manufacturing large numbers of segments.

Referring again to FIGS. 3A and 3B, in a molding process for forming the guide 26, the portion of the mold used to define the distal wall 20a includes wedge-shaped (or alternatively-shaped) mold sections around which material will deposit to form the generally v-shape channels 36a, 36b. Likewise, the portion of the mold used to define the proximal wall 32a includes wedge-shaped mold sections around which material will deposit to form the channels 48a, 48b.

FIGS. 19A and 19B schematically show that the wedge shaped mold sections M1, M2 that define the channels 36a, 36b have overlapping portions, in this case rounded apexes A1 and A2, that are longitudinally aligned with one another at overlap region O. Referring to the side elevation view of FIG. 19A, material deposits on surfaces S1 and S2 to form surfaces 28a, b (FIG. 20), respectively, but material is prevented from depositing at the overlap region. Thus when the segment is removed from the mold, guide 26 is formed between the edges of surfaces 28a, b, as best shown in the cross-section view of FIG. 20. Note that while this embodiment uses wedged-shaped mold sections with overlapping apexes to define the guides, mold sections have other shapes may be used. For example, if a guide is to be formed for a pull ribbon having a rectangular cross-section, mold sections having a generally rectangular or oval overlap region might be used.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed. Moreover, features of the various disclosed embodiment may be combined in a variety of ways to produce additional embodiments.

Any and all patents, patent applications and printed publications referred to above, including for purposes of priority, are incorporated herein by reference.

We claim:

1. A deflectable instrument shaft comprising:
a plurality of actuation elements;
a plurality of alternating first and second segments strung over the actuation elements, wherein the first segments have a different shape than the second segments.

2. The instrument shaft of claim 1, wherein each of the first segments includes an end face having a pair of planar surfaces forming a peak having a first angle, wherein the each of the second segments includes an end face having a pair of planar surfaces forming a saddle having a second angle, wherein the first angle is greater than the second angle.

3. The instrument shaft of claim 1, wherein each of the first segments has a first longitudinal length and each of the second segments has a second longitudinal length, wherein the second longitudinal length is shorter than the first longitudinal length.

4. The instrument shaft of claim 3, wherein the first and second segments are formed of different materials.

5. The instrument shaft of claim 1 wherein adjacent peaks and saddles are in contact with each other.

6. The instrument shaft of claim 1, wherein the peak defines a first longitudinal plan, and wherein each first segment includes a second end face having a pair of planar surfaces forming a second saddle defining a second longitudinal plane, the first and second longitudinal planes transverse to each other.

7. The instrument shaft of claim 6, wherein each of the second segments includes a second end face having a pair of planar surfaces forming a second peak, wherein adjacent second peaks and second saddles are in contact with each other.

8. The instrument shaft of claim 6, wherein the first and second planes are orthogonal to one another.

9. The instrument shaft of claim 1, wherein each segment includes a plurality of guides, the actuation elements extending through the guides.

10. The instrument shaft of claim 1, wherein each segment includes a plurality of channels, each channel longitudinally aligned with a corresponding guide.

11. The instrument shaft of claim 10, wherein each channel faces radially inwardly or radially outwardly.

12. The instrument shaft of claim 11, wherein a pair of channels are longitudinally aligned with each guide, each pair of channels including a radially-inwardly facing channel and a radially-outwardly facing channel.

13. The instrument shaft of claim 9, wherein at least one of the segments includes a first surface facing in a first direction, the first surface including an outer edge, a second surface facing in a second direction, the second surface including an inner edge radially aligned with the outer edge of the first surface, wherein a gap between the outer edge and the inner edge defines a guide in the segment.

14. The instrument shaft of claim 13, wherein the first and second surfaces are planar surfaces.

15. The instrument shaft of claim 15, wherein the first surface includes a first apex region extending radially outwardly and with the outer edge disposed in the first apex region, and the second surface includes a second apex region extending radially inwardly and with the inner edge disposed in the second apex region, wherein the first and second apex regions are longitudinally aligned to define the gap between the outer and inner edges.

16. A deflectable instrument shaft comprising:
a plurality of actuation elements;
a plurality of segments, each segment including a first, socket, portion having internal surface having a partially-spherical contour, and a second portion having an external surface having a partially-spherical contour, the segments strung over the actuation elements and positioned with the second portion of each segment disposed within the adjacent first portion of an adjacent segment.

17. The instrument shaft of claim 7, wherein each segment includes a first anti-rotation feature on its first portion and a second anti-rotation feature on its second portion, wherein the first anti-rotation feature on each segment is engagable with the second anti-rotation feature of an adjacent segment, the anti-rotation features when engaged preventing relative axial rotation of adjacent segments.
